(19) United States
(12) Patent Application Publication
(10) Pub. No.: US 2015/0213209 A1
(43) Pub. Date: Jul. 30, 2015

(54) Care Service Transaction Method and System Thereof

(71) Applicant: Challentech International Corporation, Zhubei City (TW)

(72) Inventors: Chung-I Chiang, Zhongli City (TW); Yun-Kuei Chiu, New Taipei City (TW)

(73) Assignee: Challentech International Corporation

(21) Appl. No.: 14/313,158

(22) Filed: Jun. 24, 2014

(30) Foreign Application Priority Data
Jan. 29, 2014 (TW) 103103386

(51) Int. Cl. G06F 19/00; H04N 5/232; H04N 7/18; G06Q 30/02; H04N 5/33

(52) U.S. Cl. CPC G06F 19/345; G06Q 30/0283; H04N 5/33; H04N 7/188; H04N 5/23206; H04N 2201/0003

(57) ABSTRACT

A care service transaction method and system thereof. The method includes the following steps: first, a thermal camera and a control apparatus are disposed in a care place. Health data of the user is then received, and a care condition is determined based on the health data, and a transaction amount is determined based on the care condition. The control apparatus is set to control the thermal camera based on the care condition, upon reception of paid information. Therefore, the user can buy suitable intelligent care service, such as care in various time periods, various areas or various care levels, upon his/her demand.

### CROSS-REFERENCE TO RELATED APPLICATION

This application claims the benefit of Taiwan Patent Application No. 103103386, filed on Jan 29, 2014, in the Taiwan Intellectual Property Office, the disclosure of which is incorporated herein in its entirety by reference.

### BACKGROUND OF THE INVENTION

1. Field of the Invention

The present disclosure generally relates to a care service transaction method, in particular, to a transaction method capable of setting different care conditions upon requirement of the cared person.

2. Description of the Related Art

As society develops maturely, people pay more attention to healthcare quality, particularly to care environment. However, traditional care environment usually requires a great deal of manpower, but the healthcare assistants are not enough for a long time. Therefore, what is need is to reduce the healthcare manpower required in care place by using an intelligent monitoring system.

In addition, the cared person must stay in the care place for a long time, and sleep or bath in the care place. Therefore, it is important that the privacy of the cared person must be protected from being infringed by the intelligent monitoring system.

An infrared thermography imager is applied to capture the thermal image of a place, and the relative relationship of temperatures of objects within the place can be determined from the thermal image. Moreover, the absolute temperature of the objects can also be determined according to the thermal image captured by the calibrated infrared thermography imager. However, at present the infrared thermography imager is only applied on the single device, and not further applied on the commercial application.

### SUMMARY OF THE INVENTION

Therefore, one of objectives of the present disclosure is to provide a care service transaction method and system thereof to reduce the required care manpower by way of intelligent monitoring.

Another objective of the present disclosure is to provide a care service transaction method and system thereof to provide user purchasing an appropriate intelligent monitoring care service upon demand, for example, the service provided at different period, different region or different care degree.

Another objective of the present disclosure is to provide a care service transaction method and system thereof capable of suggesting an appropriate intelligent monitoring service automatically according to a health status of the user.

An exemplary embodiment of the present disclosure provides a care service transaction method which comprises following steps. First, a thermal camera and a control apparatus are disposed in a care place. Upon reception of health data of the user, a care condition is determined based on the health data and a transaction amount is determined based on the care condition. The control apparatus is set to control the thermal camera based on the care condition, upon reception of the payment checking information.

Preferably, the health data comprises a medical advice, a patient record or surgical operation data.

Preferably, the care condition comprises operation of not activating thermal camera, operation of activating thermal camera whole day, operation of only activating the thermal camera for a specific period, or operation of only activating the thermal camera for a specific region.

Preferably, the care service transaction method according to the present disclosure further comprises a step of determining whether the user can stand or walk by himself or herself or has possibility of fever, in order to suggest the care condition.

Preferably, the care service transaction method according to the present disclosure further comprises a step of connecting to a medical record database to acquire the health data.

Preferably, the care place includes a ward, a nurse place, a nursery, a labor room, a child care place, a nursery or an elderly person care place.

Preferably, the payment checking information comprises information of identity document guaranty, insurance checking, cash payment, check payment, electronic money payment or credit card payment.

An exemplary embodiment of the present disclosure provides a care service transaction system which comprises a thermal camera, a control apparatus and a care setting apparatus. The thermal camera is disposed in a care place for capturing a thermal image of the care place. The control apparatus controls turn-on/off of the thermal camera, and analyses the thermal image to perform care for the user in the care place. The care setting apparatus determines a care condition according to health data of the user, and determines a transaction amount according to the care condition. After receiving a payment checking information, the care setting apparatus sets the control apparatus to control the thermal camera according to the care condition.

Preferably, the health data comprises a medical advice, a patient record or a surgical operation data.

Preferably, the control apparatus does not activate the thermal camera, or activates the thermal camera whole day, or only activates the thermal camera for a specific period, or only activates the thermal camera for a specific region, according to the care condition.

Preferably, the care setting apparatus determines whether the user can stand or walk by himself or herself or have probability of fever, to suggest the care condition.

Preferably, the care setting apparatus is connected to a medical record database to acquire the health data.

Preferably, the care place includes a ward, a nurse place, a nursery, a labor room, a child care place, a nursery or an elderly care place.

Preferably, the payment checking information comprises information of identity document guaranty, insurance checking, cash payment, check payment, electronic money payment or credit card payment.

### BRIEF DESCRIPTION OF THE DRAWINGS

The detailed structure, operating principle and effects of the present disclosure will now be described in more details hereinafter with reference to the accompanying drawings that show various embodiments of the present disclosure as follows.

FIG. 1 is a block diagram of a care service transaction system according to the present disclosure.

FIG. 2 is a flow diagram of a care service transaction method according to the present disclosure.

FIG. 3 is a flow diagram of a first embodiment of the care service transaction method according to the present disclosure.

FIG. 4 is a flow diagram of a second embodiment of the care service transaction method according to the present disclosure.

### DETAILED DESCRIPTION OF THE PREFERRED EMBODIMENTS

Reference will now be made in detail to the exemplary embodiments of the present disclosure, examples of which are illustrated in the accompanying drawings. Therefore, it is to be understood that the foregoing is illustrative of exemplary embodiments and is not to be construed as limited to the specific embodiments disclosed, and that modifications to the disclosed exemplary embodiments, as well as other exemplary embodiments, are intended to be included within the scope of the appended claims. These embodiments are provided so that this disclosure will be thorough and complete, and will fully convey the inventive concept to those skilled in the art. The relative proportions and ratios of elements in the drawings may be exaggerated or diminished in size for the sake of clarity and convenience in the drawings, and such arbitrary proportions are only illustrative and not limiting in any way. The same reference numbers are used in the drawings and the description to refer to the same or like parts.

It will be understood that, although the terms ‘first’, ‘second’, ‘third’, etc., may be used herein to describe various elements, these elements should not be limited by these terms. The terms are used only for the purpose of distinguishing one component from another component. Thus, a first element discussed below could be termed a second element without departing from the teachings of embodiments. As used herein, the term “or” includes any and all combinations of one or more of the associated listed items.

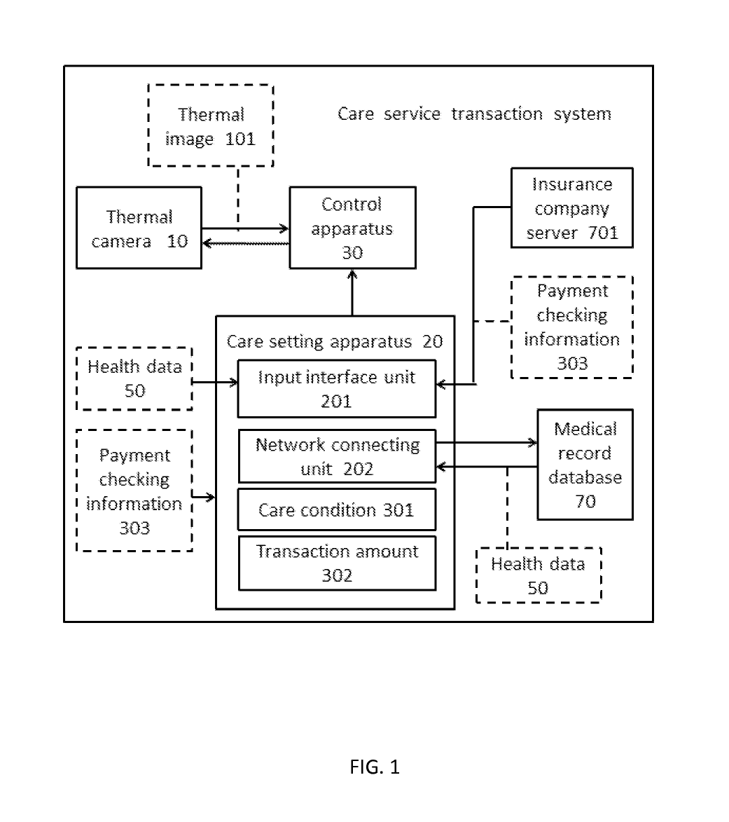

Please refer to FIG. 1 which is a block diagram of a care service transaction system according to the present disclosure. The care service transaction system comprises a thermal camera 10, a control apparatus 30, and a care setting apparatus 20. The thermal camera 10 is disposed in a care place for capturing a thermal image 101 of the care place. The thermal camera 10 is calibrated already, so each pixel within the thermal image 101 can correspond to a temperature value. In implement, the thermal camera 10 can be an infrared thermography imager, and the care place includes a ward, a nurse place, a nursery, a labor room, a child care place, a nursery or an elderly person care place.

The control apparatus 30 controls turn-on/off of the thermal camera 10, and analyses the thermal image 101 to perform care for the user in the care place. In the care place, the temperature of human body is usually higher than that of the environment, so the control apparatus 30 can first determine position of the user in the care place according to a region in which temperature is higher than a preset temperature in the thermal image 101, and then extract the temperature of the user from the region and perform care according to the temperature.

For example, if the control apparatus 30 determines that the temperature of the user increases two degrees within five minutes, it is possible that the user has a fever which is a symptom of illness, so the increasing of temperature should be defined as an abnormal event and a care assistant must be noticed. Alternatively, the temperature of the user decreases two degrees within five minutes, it is possible that vital signs of the user becomes weak, and so the decreasing of temperature also defined as the abnormal event and the care assistant must be noticed.

The care setting apparatus 20 determines a care condition 301 according to health data 50. In implementation, the care setting apparatus 20 may include an input interface unit 201 to input the health data 50 of the user, or include a network connecting unit 202 to connect with a medical record database 70 to acquire the health data 50. The health data 50 includes a medical advice, a patient record or a surgical operation data. According to care condition 301, the control apparatus 30 does not activate the thermal camera 10, or activates the thermal camera 10 whole day, or only activates the thermal camera 10 for a specific period, or only activates the thermal camera 10 for a specific region. Besides, the control apparatus 30 may also set the capturing frequency for the thermal camera 10 to capture the thermal image 101 according to the care condition 301. The higher capturing frequency indicates higher care degree which is suitable for patient who is just performed an operation or has unstable body status.

The ward and patient are taken as example of the care place and the user for following illustration. After receiving a treatment, such as surgical treatment or fracture treatment, the patient must stay in hospital for a long time. When the nurse selects a ward for this patient, the nurse can operate the care setting apparatus 20 to set the care condition 301 of the ward. First, the nurse can acquire the medical advice of the patient from the medical record database 70 of the hospital by the network connecting unit 202. According to the medical advice, the nurse determines whether the patient can stand or walk by himself or herself or has possibility of fever.

If the patient can walk and does not have possibility of fever, the nurse can set the care condition 301 as the operation of not activating the thermal camera 10. If the patient can walk by himself or herself but may fever during night, the nurse can set the care condition 301 as the operation of only activating the thermal camera 10 for a specific period from 10 pm to 8 am; if the user can walk and probably fever, the nurse can set the care condition 301 as the operation of activating the thermal camera 10 whole day. If the ward is not a single room, the nurse can set the care condition 301 as the operation of only activating the thermal camera 10 for a specific region, or only analyzing a specific region in the thermal image 101.

After the thermal camera 10 is activated, the control apparatus 30 can monitor the temperature of the user continuously, and the user does not have discomfort caused by wearing a contact-type thermometer. Besides, the nurse does not need to visit ward for measuring temperature frequently, so the care assistant manpower can be saved efficiently.

In addition, after receiving the health data 50, the care setting apparatus 20 may also suggest the care condition 301 automatically according to whether the user can stand or walk by himself or herself or has possibility of fever, if necessary.

The child care center and child are taken as example of the care place and the user for illustration. When leaving a child in the child care center, the parent can input the health data 50 of the child into the care setting apparatus 20, such as possibility of fever, capability of walking by himself or herself, in order to determine the care condition 301. The feature of the thermal camera 10 is in measuring temperature by contactless way, so the child can move free in the child care center. The parent can set degree of the care condition 301.

After the care condition 301 is set completely, the care setting apparatus 20 determines a transaction amount 302 according to the care condition 301. For example, the price for activating the thermal camera 10 all day is larger than that of only activating the thermal camera 10 in specific period, however, the present disclosure is not limited thereto.

Finally, after the care setting apparatus 20 receives a payment checking information 303, the care setting apparatus 20 sets the control apparatus 30 to control the thermal camera 10 according to the care condition 301. Preferably, the payment checking information 303 may comprise information of identity document guaranty, insurance checking, cash receiving, check receiving, electronic money payment or alternatively credit card payment. For example, after receiving a national health insurance card, cash, check or electronic money payment from the user, the nurse input a payment checking information 303 in the care setting apparatus 20. Alternatively, the use inputs insurance data, and the care setting apparatus 20 is connected to an insurance company server 701 automatically, and the care setting apparatus 20 set the control apparatus 30 upon receipt of the payment checking information 303 from the insurance company server 701.

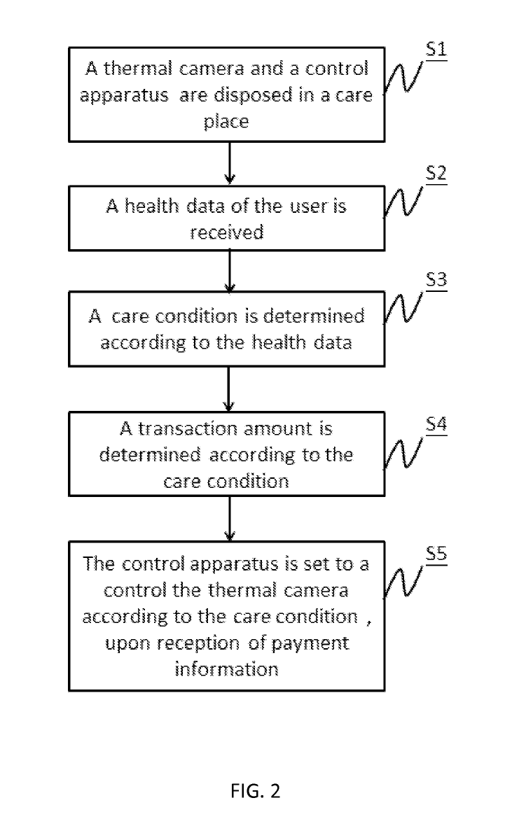

Please refer to FIG. 2 which is a flow diagram of a care service transaction method according to the present disclosure. The care service transaction method is cooperated with the block diagram of FIG. 1 for illustration, and comprises following steps. In step S1, a thermal camera 10 and a control apparatus 30 are disposed in a care place. In implementation, the care place includes a ward, a nurse place, a nursery, a labor room, a child care place, a nursery or an elderly care place.

Next, in step S2, health data 50 of the user is received. In implementation, the health data 50 can be inputted by the user or an operator, or can be obtained by connecting to the medical record database 70 of the hospital. The health data 50 includes a medical advice, a patient record or surgical operation data.

In step S3, a care condition 301 is determined according to the health data 50. The care condition 301 includes the operation of not activating the thermal camera 10, or activating the thermal camera 10 all day, or only activating the thermal camera 10 for a specific period, or only activating the thermal camera 10 for a specific region.

In step S4, a transaction amount 302 is determined according to the care condition 301. In step S5, the control apparatus 30 is set to a control the thermal camera 10 according to the care condition 301, upon reception of paid information 303.

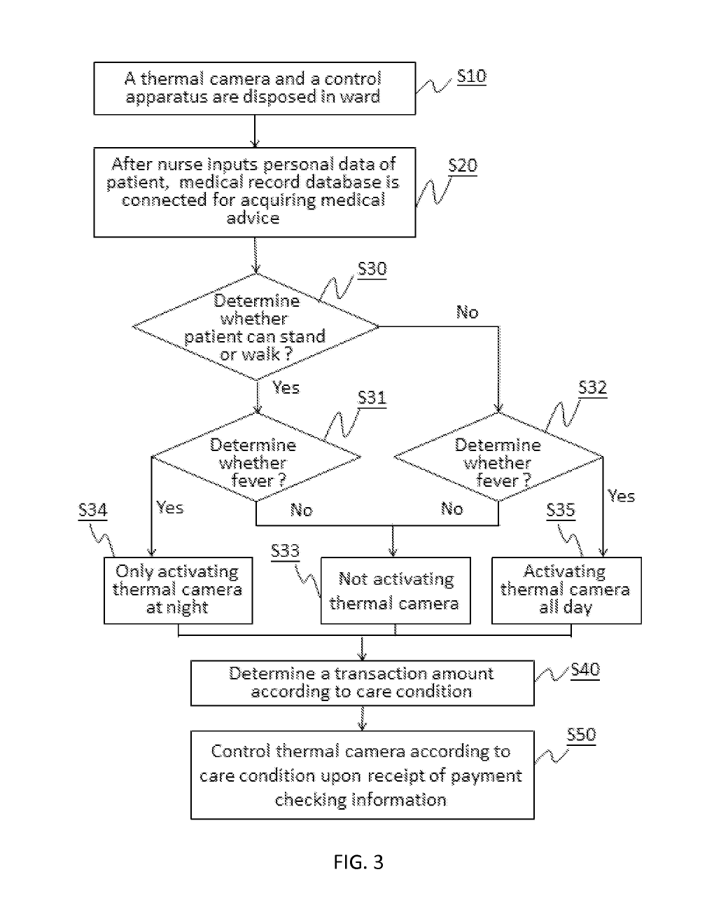

Please refer to FIG. 3 which is a flow diagram of a first embodiment of the care service transaction method according to the present disclosure. The ward and patient are taken as example of the care place and the user for illustration. In step S10, a thermal camera 10 and a control apparatus 30 are disposed in ward. In step S20, after the nurse operates the care setting apparatus 20 to input personal data of the patient, the medical record database 70 of the hospital is connected for acquiring a medical advice of the patient. In step S30, the nurse determines whether the patient can stand or walk by himself/herself according to the medical advice; if yes, the step S31 is executed; otherwise, if no, the step 32 is executed.

In step S31, the nurse determines whether the patient has possibility of fever according to the medical advice; if no, the care condition 301 is set as not activating the thermal camera 10 in step S33; if yes, the care condition 301 is set as only activating the thermal camera 10 at night in step S34.

In step S32, the nurse determines whether the patient probably fevers, according to the medical advice; if no, the step S33 is executed; if yes, the care condition 301 is set as the operation of activating the thermal camera 10 all day in step S35. In addition, if the patient probably fevers, the nurse can set capturing frequency of the thermal image 101 of the thermal camera 10 according to degree of probability of fever. The higher capturing frequency is set for higher possibility of fever, to strengthen the care degree.

In step S40, after the care setting apparatus 20 determines a transaction amount 302 correspondingly according to the care condition 301 determined by the nurse, the nurse can notify the user or family for payment. Finally, in step S50, after receiving a payment checking information 303 which indicates the user or family pay already, the nurse sets the control apparatus 30 to control the thermal camera 10 according to the care condition 301.

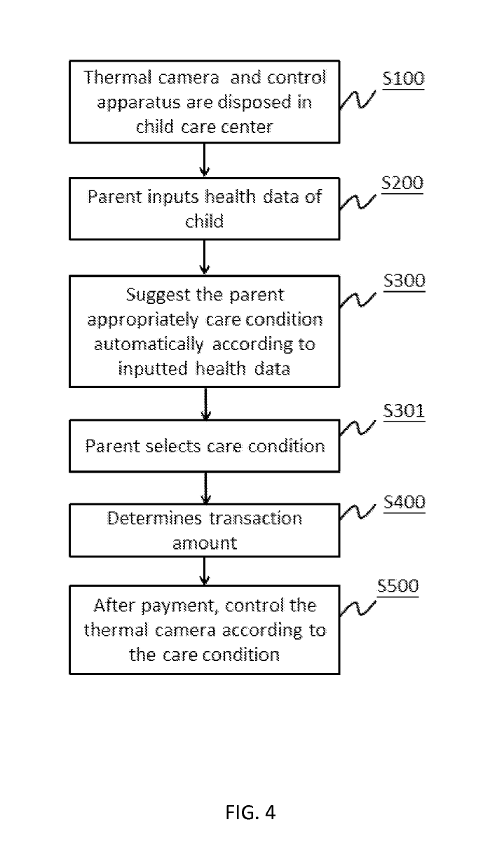

Please refer to FIG. 4 which is a flow diagram of a second embodiment of a care service transaction method according to the present disclosure. The child care center and child are taken as example of the care place and the user for illustration. In step S100, a thermal camera 10 and a control apparatus 30 are disposed in a child care center. In step S200, the parent operates the care setting apparatus 20 to input the health data 50 of child, such as whether the child fevers. In step S300, the care setting apparatus 20 suggests the parent an appropriately care condition 301 automatically according to the inputted health data 50. For example, the suggested appropriately care condition 301 includes the operation of activating the thermal camera 10 or controlling the thermal camera 10 to measure temperature of the child by different capturing frequency.

In step S301, the parent selects the care condition 301 in the care setting apparatus 20. In step S400, the care setting apparatus 20 determines and displays transaction amount 302 correspondingly. Finally, after the parent completes the payment by credit card or cash, the control apparatus 30 is set to control the thermal camera 10 to perform care on the child according to the care condition 301 in step 500.

The above-mentioned descriptions represent merely the exemplary embodiment of the present disclosure, without any intention to limit the scope of the present disclosure thereto. Various equivalent changes, alternations or modifications based on the claims of present disclosure are all consequently viewed as being embraced by the scope of the present disclosure.

What is claimed is:

1. A care service transaction method, comprising:
disposing a thermal camera and a control apparatus in a care place;
receiving a health data of a user;
determining a care condition according to the health data;
determining a transaction amount according to the care condition; and
setting the control apparatus to control the thermal camera according to the care condition, upon reception of a payment checking information.

2. The care service transaction method according to claim 1, wherein the health data comprise a medical advice, a patient record or a surgical operation data.

3. The care service transaction method according to claim 1, wherein the care condition comprise the operation of not activating the thermal camera, the operation of activating the thermal camera whole day, the operation of only activating the thermal camera for a specific period, or the operation of only activating the thermal camera for a specific region.

4. The care service transaction method according to claim 1, further comprising a step of determining whether the user can stand or walk by himself or herself, or have possibility of fever, to suggest the care condition.

5. The care service transaction method according to claim 1, further comprising a step of connecting to a medical record database to acquire the health data.

6. The care service transaction method according to claim 1, wherein the care place includes a ward, a nurse place, a nursery, a labor room, a child care place, a nursery or an elderly care place.

7. The care service transaction method according to claim 1, wherein the payment checking information comprises information of identity document guaranty, insurance checking, cash payment, check payment, electronic money payment or credit card payment.

8. A care service transaction system, comprising:
a thermal camera, configured for capturing a thermal image of a care place;
a control apparatus, configured for controlling turn-on/turn-off of the thermal camera, and analyzing the thermal image to perform care for a user in the care place; and
a care setting apparatus, configured for determining a care condition according to health data, and determining a transaction amount according to the care condition;
wherein after the care setting apparatus receives a payment checking information, the care setting apparatus sets the control apparatus to control the thermal camera according to the care condition.

9. The care service transaction system according to claim 8, wherein the health data comprise a medical advice, a patient record or a surgical operation data.

10. The care service transaction system according to claim 8, wherein the control apparatus does not activate the thermal camera, or activates the thermal camera whole day, or only activates the thermal camera for specific period, or only activates the thermal camera for a specific region, according to the care condition.

11. The care service transaction system according to claim 8, wherein the care setting apparatus determines whether the user can stand or walk by himself or herself, or has possibility of fever, to suggest the care condition.

12. The care service transaction system according to claim 8, wherein the care setting apparatus is connected to a medical record database to acquire the health data.

13. The care service transaction system according to claim 8, wherein the care place includes a ward, a nurse place, a nursery, a labor room, a child care place, a nursery or an elderly care place.

14. The care service transaction system according to claim 8, wherein the payment checking information comprises information of identity document guaranty, insurance checking, cash payment, check payment, electronic money payment or credit card payment.
